(19) United States
(12) Patent Application Publication
(10) Pub. No.: US 2021/0301249 A1
(43) Pub. Date: Sep. 30, 2021

(54) Compositions and Methods for Inhibiting Growth of Lactic Acid Producing Bacteria

(71) Applicant: University of Kentucky Research Foundation, Lexington, KY (US)

(72) Inventors: Jian Shi, Lexington, KY (US); Ryan M. Kalinoski, Oak Forest, IL (US)

(21) Appl. No.: 17/219,111

(22) Filed: Mar. 31, 2021

Related U.S. Application Data
(60) Provisional application No. 63/002,501, filed on Mar. 31, 2020.

(51) Int. Cl. C12N 1/20; C12P 7/06

(52) U.S. Cl. CPC C12N 1/20; C12N 2500/30; C12P 7/06

(57) ABSTRACT

The present disclosure includes a composition and a method for inhibiting bacteria growth. In particular, the present disclosure includes a composition for inhibiting lactic acid bacteria growth in a media contaminated by, or at risk of being contaminated by, one or more species of lactic acid bacteria. The composition comprises a decomposition product, such as a bio-oil, derived from an oxidative depolymerized lignin sample and includes one or more phenolic constituents. The method includes implementing the decomposition product into a media contaminated by, or at risk of being contaminated by, lactic acid bacteria in a concentration to inhibit growth of one or more species of lactic acid bacteria in the media.

### RELATED APPLICATIONS

This application claims the benefit of U.S. Provisional Patent Application Ser. No. 63/002,501, filed Mar. 31, 2020, the entire disclosure of which is incorporated herein by this reference.

### GOVERNMENT INTEREST

This invention was made with government support under grant numbers OIA-1355438 and OIA-1632854 awarded by the National Science Foundation (NSF). The government has certain rights in the invention.

### TECHNICAL FIELD

The presently-disclosed subject matter relates to a composition and a method for inhibiting bacteria growth. In particular, the presently-disclosed subject matter relates to a composition and a method which inhibit lactic acid bacteria growth in a media contaminated by, or at risk of being contaminated by, one or more species of lactic acid bacteria, and thus may prove useful in fuel ethanol fermentation processes.

### BACKGROUND

Fuel ethanol is commonly derived through fermentation processes which utilize yeast to ferment biomasses with high starch and sugar content. Such fermentations, however, do not typically occur under completely aseptic conditions. As a result, acute and chronic bacterial contaminations that negatively affect yeast growth and subsequent ethanol production are common. In particular, lactic acid bacteria (LAB) are especially problematic as LABs produce by-products, such as acetic and lactic acids, polysaccharides, and gummy biofilms, which reduce yeast viability. In this regard, proliferation of LABs in fermentation environments, such as fermentation reactors, can limit essential micronutrients and sugar required for optimal yeast growth and ethanol production. Accordingly, LAB contamination can thus reduce ethanol yields and result in “stuck” fermentations causing costly shutdowns to clean and eradicate the contamination.

To reduce occurrences of bacterial contamination, antibiotics, such as virginiamycin, penicillin, and erythromycin, are commonly introduced into the fermentation process. However, due to the overuse of antibiotics, there is an increased incidence of antibiotic resistant bacteria strains. As certain antibiotics utilized to guard against bacterial contamination, like penicillin and erythromycin, are also utilized to medically treat humans, the use of such antibiotics can promote or result in antibiotic-resistant bacterial strains posing a risk to human health. Accordingly, there is a need for compositions and methods for inhibiting LAB in fuel ethanol fermentations which reduce or eliminate the need for traditional antibiotics.

Further features and advantages of the presently-disclosed subject matter will become evident to those of ordinary skill in the art after a study of the description, figures, and non-limiting examples in this document.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and, in many cases, lists variations and permuations of these embodiments. The summary is merely exemplary of the numerous varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations.

The presently-disclosed subject matter includes a composition for inhibiting LAB growth in a media contaminated by, or at risk of being contaminated by, one or more species of LAB. The composition comprises a decomposition product that is derived from an oxidative depolymerized lignin sample and includes one or more phenolic constituents.

In some embodiments, the decomposition product is a bio-oil which includes one or more phenolic monomers derived from lignin depolymerization. In some embodiments, the lignin sample is derived from corn stover. In some embodiments, the lignin sample is depolymerized using peracetic acid. In some embodiments, the decomposition product includes at least one of Hydroquinone, p-Coumaric acid, 2,6-Dimethoxyhydroquinone, Syringic acid, Phloroglucinol, 4-Hydroxybenzaldehyde, 4-Hydroxyacetophenone, Ferulic acid, 3-Ethylphenol, 2-Hydroxybenzyl alcohol. In some embodiments, the total phenolic content of the decomposition product is at least 10 wt % of an overall weight of the decomposition product.

In some embodiments, the phenolic content of the decomposition product is sufficient to inhibit the growth of one or more species of LAB within the media by at least 30% when the decomposition product is added to the media in a concentration of at least 2 mg/ml. In one such embodiment, the one or more species of LAB includes at least one of Lactobacillus plantarum, Lactobacillus amylovorus, Lactobacillus fermentum, Pediococcus pentosaceus, Enterococcus faecalis, Bacillus amyloliquefaciens, and Acetobacter pasteurianus.

In some embodiments, the phenolic content of the decomposition product is sufficient to inhibit the growth of Lactobacillus fermentum within the media by at least 70% when the decomposition product is added to the media in a concentration of at least 1 mg/ml. In some embodiments, the phenolic content of the decomposition product is such that the decomposition product does not inhibit the growth of a species of yeast, such as Saccharomyces cerevisiae, within the media when added to the media in a concentration of 4 mg/ml or less. As Lactobacillus fermentum has been found to be one of the most prolific strains causing stuck fermentations in the fuel corn ethanol industry and yeast, such as Saccharomyces cerevisiae, is commonly used in ethanol fermentation, the composition of the present disclosure may thus find utility in ethanol fuel fermentation processes in which the inhibition of LAB within a target media is desirable to eliminate or reduce the need for the use of traditional antibiotics.

The presently-disclosed subject matter also includes a method of inhibiting bacterial growth in a media contaminated by, or at risk of being contaminated by, bacteria. In some embodiments, the method includes implementing the above-described decomposition product into a media contaminated by, or at risk of being contaminated by, lactic acid bacteria in a concentration sufficient to inhibit growth of one or more species of lactic acid bacteria within the media.

In some embodiments, the one or more species of lactic acid bacteria includes at least one of Lactobacillus plantarum, Lactobacillus amylovorus, Lactobacillus fermentum, Pediococcus pentosaceus, Enterococcus faecalis, Bacillus amyloliquefaciens, and Acetobacter pasteurianus. In one such embodiment, the decomposition product is added to the media in a concentration sufficient to inhibit the growth of the one or more species of lactic acid bacteria by at least 30%.

In some embodiments, the media to which the decomposition product is added includes a species of yeast and the decomposition product is added to the media in a concentration which does not inhibit growth of the species of yeast. In some embodiments, the media to which the decomposition product is added includes Lactobacillus fermentum and the decomposition product is added in a concentration to inhibit the growth of Lactobacillus fermentum within the media by at least 70%.

In some embodiments, the media to which the decomposition product is added is utilized in a fuel ethanol fermentation process.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIG. 1 is a heat map showing the degree of inhibition against different species of LAB and a yeast species against different concentrations of extracted bio-oil, wherein inhibition represents percent decrease in bacterial growth as compared to control without bio-oil and having a 5% ethanol concentration. At concentrations of 2-4 mg/ml, the extracted bio-oil inhibited the growth of various LABs by 75-96% and did not inhibit Saccharomyces cerevisiae (yeast).

FIG. 2 is a heat map showing the degree of inhibition against different bacterial species from the Bacillus, Escherichia, Lactobacillus, and Staphylococcus genera against different concentration of extracted bio-oil, wherein inhibition represents percent decrease in bacterial growth as compared to control without bio-oil having a 5% ethanol concentration.

### DESCRIPTIONS OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter is illustrated by specific but non-limiting examples throughout this description. The examples may include compilations of data that are representative of data gathered at various times during the course of development and experimentation related to the present invention(s). Each example is provided by way of explanation of the present disclosure and is not a limitation thereon. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

All combinations of method or process steps as used herein can be performed in any order, unless otherwise specified or clearly implied to the contrary by the context in which the referenced combination is made.

While the following terms used herein are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the disclosure belongs. Any methods and materials similar to or equivalent to those described herein can be used in the practice or testing of the present disclosure, including the methods and materials that are described below.

Following long-standing patent law convention, the terms “a,” “an,” and “the” refer to “one or more” when used in this application, including the claims. Thus, for example, reference to “a cell” includes a plurality of cells, and so forth.

The terms “comprising,” “including,” and “having” are intended to be inclusive and mean that there may be additional elements other than the listed elements.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration, percentage, or the like is meant to encompass variations of in some embodiments ±50%, in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, ElZ specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used herein, the terms “optional” or “optionally” means that the subsequently described event or circumstance can or cannot occur, and that the description includes instances where said event or circumstance occurs and instances where it does not.

The term “lactic acid bacteria” refers to gram-positive bacteria which produce lactic acid as an end product of carbohydrate fermentation.

As will be recognized by one of ordinary skill in the art, the term “inhibit” or grammatical variations thereof do not necessarily refer to the ability to completely prevent a target biological activity, such as the growth of one or more species of lactic acid bacteria, from occurring in all cases. Rather, the skilled artisan will understand that such terms can also, in some embodiments, refer to decreasing or limiting the extent to which the target biological activity continues. The prevention, decrease, or limitation of the target biological activity can be determined relative to a control, wherein an inhibitor, such as a composition described herein, is not administered in an environment where the target biological activity is occurring or is likely to occur, such as in a media contaminated by, or likely to be contaminated by, one or more species of lactic acid bacteria. For example, in some embodiments, the decrease or limitation relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

A composition for inhibiting lactic acid bacteria (LAB) growth in a media contaminated by, or at risk of being contaminated by, one or more species of LAB is provided herein. The composition of the present disclosure may find utility in certain applications, including, but not limited to, fuel ethanol fermentation process, in which the inhibition of LAB within a target media is desirable to eliminate or reduce the need for the use of traditional antibiotics in such applications.

The composition comprises a decomposition product that is derived from an oxidative depolymerized lignin sample and includes one or more phenolic constituents. More specifically, the decomposition product is a bio-oil which includes one or more phenolic monomers derived from lignin depolymerization.

In some embodiments, the bio-oil is a mixture of phenolic monomers derived from lignin depolymerization. In some embodiments, the phenolic content of the bio-oil includes one or more hydroxylated and/or acidic phenolic compounds derived from lignin. For example, in some embodiments, the bio-oil includes phenolic monomer compounds including at least one of Hydroquinone, p-Coumaric acid, 2,6-Dimethoxyhydroquinone, Syringic acid, Phloroglucinol, 4-Hydroxybenzaldehyde, 4-Hydroxyacetophenone, Ferulic acid, 3-Ethylphenol, 2-Hydroxybenzyl alcohol, and combinations thereof (Table 4). In some embodiments, Hydroquinone has the highest yield of the phenolic monomers present within the bio-oil (Table 4). In some embodiments, the phenolic monomers comprise at least 1% of the total weight of the bio-oil (i.e., 1 wt % of the bio-oil) (Table 4). In some embodiments, the phenolic monomer compounds comprise about 1.8% of the total weight of the bio-oil (i.e., 1.8 wt % of the bio-oil) (Table 4).

Additionally or alternatively, in some embodiments, the bio-oil also includes one or more larger oligomers derived from the depolymerization of lignin that have a molecular weight ranging from about 400 Daltons to about 35,000 Daltons. In some embodiments, the average weight of the larger oligomers present in the bio-oil may be about 2,000 Daltons.

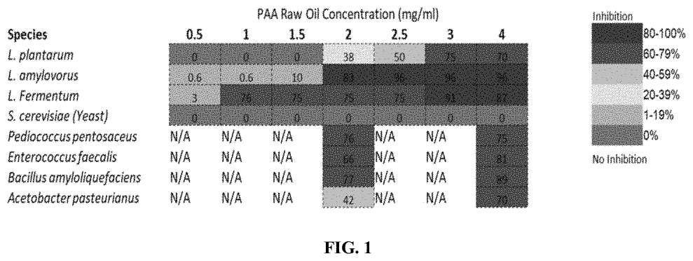

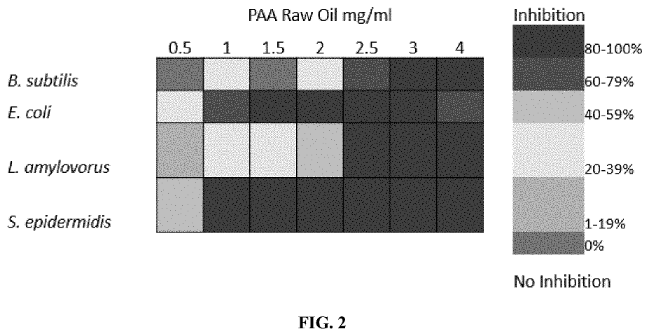

The phenolic content of the bio-oil inhibits the growth of one or more species of LAB. Surprisingly, however, unlike depolymerized lignin products derived from other depolymerization methods that also inhibit yeast, the bio-oil disclosed herein does not inhibit the growth of a species of yeast commonly used in ethanol fermentation, such as Saccharomyces cerevisiae. Accordingly, in some implementations, the bio-oil disclosed herein may be implemented into a media utilized in ethanol fermentation processes to inhibit the growth of bacteria without also inhibiting the growth of a yeast species commonly utilized therein (e.g., Saccharomyces cerevisiae) (FIG. 1). LABs which may be inhibited by the bio-oil include Lactobacillus plantarum, Lactobacillus amylovorus, Lactobacillus fermentum, Pediococcus pentosaceus, Enterococcus faecalis, Bacillus amyloliquefaciens, and Acetobacter pasteurianus. In some embodiments, the phenolic content of the bio-oil is also sufficient to inhibit the growth of one or more species from the Bacillus, Escherichia, and Staphylococcus genera (FIG. 2) when added in sufficient concentrations.

In some embodiments, the total phenolic content of the bio-oil is at least 10% of the bio-oil's total weight (i.e., 10 wt % of the bio-oil). In some embodiments, the phenolic content of the bio-oil is about 12% or greater of the bio-oil's total weight (i.e., 12 wt % of the bio-oil). In some embodiments, the phenolic content of the bio-oil is sufficient to inhibit the growth of Lactobacillus fermentum in a media containing the same by at least 70% when the bio-oil is added to a media containing such bacteria in a concentration of at least 1.0 mg/ml (FIG. 1). In some embodiments, the phenolic content of the bio-oil is also sufficient to inhibit the growth of Lactobacillus plantarum, Lactobacillus amylovorus, Lactobacillus fermentum, Pediococcus pentosaceus, Enterococcus faecalis, Bacillus amyloliquefaciens, and Acetobacter pasteurianus by at least 30% when the bio-oil is added thereto in a concentration of at least 2.0 mg/ml (FIG. 1). In some embodiments, the phenolic content of the bio-oil is sufficient to inhibit the growth of Escherichia coli and Staphylococcus epidermidis by at least 60% when the bio-oil is added to a media containing the same in a concentration of at least 1.0 mg/ml (FIG. 2). In some embodiments, the phenolic content of the bio-oil is sufficient to inhibit the growth of Bacillus subtilis by at least 60% when added to a media containing the same in a concentration of at least 2.5 mg/ml (FIG. 2).

In some embodiments, the bio-oil of the present disclosure also does not impede the enzymatic function of α-amylase and glucoamylase during saccharification when added in concentrations of at least 4 mg/ml (Tables 5 and 6).

Also provided herein is a method of synthesizing the above-described bio-oil. The method includes depolymerizing a lignin sample by reacting the sample with one or more chemical oxidizers and subsequently extracting the bio-oil from the reaction. The lignin sample from which the bio-oil is derived is extracted from macrofibrils found in the cell wall of plant life. Accordingly, the source of the lignin sample may be selected from a number of plant-based sources. As lignocellulose biomasses are commonly used in ethanol production, the plant matter from which the lignocellulose is derived is processed to separate the lignocellulose from the lignin, so that the sugar fraction can be fermented into ethanol. Thus, in some embodiments, the lignin sample referred to herein may be obtained from, or be a waste product of, biofuel production processes currently utilized within the art. In one embodiment, the lignin sample may be alkaline pretreated lignin from corn stover.

Although discussed herein primarily with respect to lignin from corn stover, as will be appreciated by those skilled in the art, the disclosure is not so limited and may include lignin from any other suitable source. Other suitable sources include, but are not limited to, Kraft, organosolv, steam explosion, dilute acid, alkali, soda, and klason lignins, or lignosulfonates derived from hardwood, softwood, or grasses. As will also be appreciated by those skilled in the art, the concentration of phenolic monomers resulting from the depolymerization of lignin may differ based upon the lignin source. Accordingly, in some embodiments, the concentration of the resulting bio-oil sufficient to provide LAB inhibition may also vary depending upon the lignin source utlized.

In some embodiments, once the lignin sample is obtained, the lignin sample is preferably analyzed for purity prior to depolymerization by one or more chemical oxidizers. In some embodiments, such as where the lignin contains excess residual sugars, the lignin sample can be subjected to a precipitation purification process, as further described below with reference to Tables 1 and 2. A high purity lignin, preferably greater than 90 wt %, is important as residual sugars may be converted into furan-based compounds (i.e. furfural) that are inherently antimicrobial, especially towards yeast. In some embodiments, the lignin sample may be purified until the sample is at least 95 wt % of the sample (Table 2).

Once the lignin sample is of a desired purity, the sample is depolymerized using one or more chemical oxidizers. Suitable chemical oxidizers include, but are not limited to, peracetic acid, peroxymonosulfuric acid, hydrogen peroxide, sodium peroxide, any other suitable peroxygen chemical, and combinations thereof. For example, in one embodiment, peracetic acid is utilized as the sole chemical oxidizer. In such embodiments, the lignin sample is subjected to a peracetic acid (PAA) treatment in which the lignin sample is mixed with PAA and diluted. In some embodiments, the lignin sample may be mixed with PAA at a 0.2-1 g lignin/g PAA concentration with 5% solid loading (diluted with water or glacial acetic acid). The bio-oil is subsequently extracted from the reaction mixture. In some embodiments, the bio-oil may be extracted using ethyl acetate. In other embodiments, the bio-oil may be extracted by way of nitrogen evaporation.

A method of inhibiting bacterial growth in a media contaminated by, or at risk of being contaminated by, bacteria is also provided herein. In some embodiments, the method includes implementing the bio-oil disclosed herein into a media containing, or at risk of containing, LAB in an amount sufficient to inhibit growth of one or more species of LAB.

In some embodiments, the one or more species of LAB includes at least one of Lactobacillus plantarum, Lactobacillus amylovorus, Lactobacillus fermentum, Pediococcus pentosaceus, Enterococcus faecalis, Bacillus amyloliquefaciens, and Acetobater pasteurians. In some embodiments, the bio-oil is implemented in the media in a concentration of 1 mg/ml, 1.5 mg/ml, 2 mg/ml, 3 mg/ml, or 4 mg/ml. In one embodiment, for example, the method includes implementing the bio-oil into the contaminated media in a concentration sufficient to inhibit the growth of one or more species of LAB by at least 10%. In another embodiment, the method includes implementing the bio-oil into the bacterially contaminated media in a concentration sufficient to inhibit growth of one or more species of LAB by at least 20%. In another embodiment, the method includes implementing the bio-oil into the bacterially contaminated media in a concentration sufficient to inhibit growth of one or more species of LAB by at least 30%. In another embodiment, the method includes implementing the bio-oil into the bacterially contaminated media in a concentration sufficient to inhibit growth of one or more species of LAB by at least 40%. In another embodiment, the method includes implementing the bio-oil into the bacterially contaminated media in a concentration sufficient to inhibit growth of one or more species of LAB by at least 50%. In another embodiment, the method includes implementing the bio-oil into the bacterially contaminated media in a concentration sufficient to inhibit growth of one or more species of LAB by at least 60%. In another embodiment, the method includes implementing the bio-oil into the bacterially contaminated media in a concentration sufficient to inhibit growth of one or more species of LAB by at least 70%. In another embodiment, the method includes implementing the bio-oil into the bacterially contaminated media in a concentration sufficient to inhibit growth of one or more species of LAB by at least 80%. In another embodiment, the method includes implementing the bio-oil into the bacterially contaminated media in a concentration sufficient to inhibit growth of one or more species of LAB by at least 90%.

In some embodiments, the method includes implementing the bio-oil disclosed herein into a media containing one or more species of bacteria from the Bacillus genus. In one embodiment, for example, the method includes implementing the bio-oil into the contaminated media in a concentration sufficient to inhibit the growth of Bacillus subtilis genus by at least 10%. In another embodiment, the method includes implementing the bio-oil into the bacterially contaminated media in a concentration sufficient to inhibit growth of Bacillus subtilis by at least 20%. In another embodiment, the method includes implementing the bio-oil into the bacterially contaminated media in a concentration sufficient to inhibit growth of Bacillus subtilis by at least 30%. In another embodiment, the method includes implementing the bio-oil into the bacterially contaminated media in a concentration sufficient to inhibit growth of Bacillus subtilis by at least 40%. In another embodiment, the method includes implementing the bio-oil into the bacterially contaminated media in a concentration sufficient to inhibit growth of Bacillus subtilis by at least 50%. In another embodiment, the method includes implementing the bio-oil into the bacterially contaminated media in a concentration sufficient to inhibit growth of Bacillus subtilis by at least 60%. In another embodiment, the method includes implementing the bio-oil into the bacterially contaminated media in a concentration sufficient to inhibit growth of Bacillus subtilis by at least 70%. In another embodiment, the method includes implementing the bio-oil into the bacterially contaminated media in a concentration sufficient to inhibit growth of Bacillus subtilis by at least 80%.

In some embodiments, the method includes implementing the bio-oil disclosed herein into a media containing one or more species of bacteria from the Escherichia genus. In one embodiment, for example, the method includes implementing the bio-oil into the contaminated media in a concentration sufficient to inhibit the growth of Escherichia coli by at least 10%. In another embodiment, the method includes implementing the bio-oil into the bacterially contaminated media in a concentration sufficient to inhibit growth of Escherichia coli by at least 20%. In another embodiment, the method includes implementing the bio-oil into the bacterially contaminated media in a concentration sufficient to inhibit growth of Escherichia coli by at least 30%. In another embodiment, the method includes implementing the bio-oil into the bacterially contaminated media in a concentration sufficient to inhibit growth of Escherichia coli by at least 40%. In another embodiment, the method includes implementing the bio-oil into the bacterially contaminated media in a concentration sufficient to inhibit growth of Escherichia coli by at least 50%. In another embodiment, the method includes implementing the bio-oil into the bacterially contaminated media in a concentration sufficient to inhibit growth of Escherichia coli by at least 60%. In another embodiment, the method includes implementing the bio-oil into the bacterially contaminated media in a concentration sufficient to inhibit growth of Escherichia coli by at least 70%. In another embodiment, the method includes implementing the bio-oil into the bacterially contaminated media in a concentration sufficient to inhibit growth of Escherichia coli by at least 80%.

In some embodiments, the method includes implementing the bio-oil disclosed herein into a media containing one or more species of bacteria from the Staphylococcus genus. In one embodiment, for example, the method includes implementing the bio-oil into the contaminated media in a concentration sufficient to inhibit the growth of Staphylococcus epidermidis by at least 10%. In another embodiment, the method includes implementing the bio-oil into the bacterially contaminated media in a concentration sufficient to inhibit growth of Staphylococcus epidermidis by at least 20%. In another embodiment, the method includes implementing the bio-oil into the bacterially contaminated media in a concentration sufficient to inhibit growth of Staphylococcus epidermidis by at least 30%. In another embodiment, the method includes implementing the bio-oil into the bacterially contaminated media in a concentration sufficient to inhibit growth of Staphylococcus epidermidis by at least 40%. In another embodiment, the method includes implementing the bio-oil into the bacterially contaminated media in a concentration sufficient to inhibit growth of Staphylococcus epidermidis by at least 50%. In another embodiment, the method includes implementing the bio-oil into the bacterially contaminated media in a concentration sufficient to inhibit growth of Staphylococcus epidermidis by at least 60%. In another embodiment, the method includes implementing the bio-oil into the bacterially contaminated media in a concentration sufficient to inhibit growth of Staphylococcus epidermidis by at least 70%. In another embodiment, the method includes implementing the bio-oil into the bacterially contaminated media in a concentration sufficient to inhibit growth of Staphylococcus epidermidis by at least 80%.

In some embodiments, the method includes implementing the bio-oil into a media containing yeast, such as Saccharomyces cerevisiae. In such embodiments, the bio-oil is preferably added to the media in a concentration which does not inhibit yeast growth. In one such embodiment, the bio-oil is added to the media in a concentration of 4 mg/ml or less.

In some embodiments, the method includes implementing the bio-oil into a media containing Lactobacillus fermentum. In such embodiments, the bio-oil is preferably added to the media in a concentration which inhibits Lactobacillus fermentum by at least 70%. In one such embodiment, the bio-oil is added to the media in a concentration of at least 1 mg/ml.

In some embodiments, the media to which the bio-oil is implemented is a media utilized in a fuel ethanol fermentation process.

The bio-oil disclosed herein may be implemented into the media in any suitable form. For example, in some embodiments, the method includes implementing the bio-oil into the media as an emulsion by suspending the bio-oil in an aqueous mixture of alcohol and water. In alternative embodiments, the bio-oil may be lyophilized into a solid medium (e.g., dried particles) and then implemented into the media. In some implementations, the media to which the bio-oil is implemented may be a media utilized in fuel ethanol fermentation.

The presently-disclosed subject matter is further illustrated by the following specific but non-limiting example. The following example may include compilations of data that are representative of data gathered at various times during the course of development and experimentation related to the presently-disclosed subject matter. Those skilled in the art will recognize, or be able to ascertain, using no more than routine experimentation, numerous equivalents to the specific substances and procedures described herein.

### Example

This Example focuses on the discovery that bio-oil derived from oxidative depolymerized lignin can be used to effectively inhibit the growth of LABs, some of which are known to adversely affect fuel ethanol fermentation processes. In particular, the following example shows that that lignin derived from a corn stover biomass can be oxidatively depolymerized using a PAA treatment to extract bio-oil having a phenolic content sufficient to enable the bio-oil to effectively act as an antimicrobial against different bacterial species. Importantly, the following example shows that such bio-oil can be implemented into a LAB-contaminated media to inhibit the growth of one or more species of LAB without also inhibiting yeast growth and/or hindering α-amylase or glucoamylase enzymatic saccharification activity. In view of the foregoing, the bio-oil of the present disclosure thus has potential act as an antibiotic substitute in fuel ethanol fermentations in which LAB contaminations are common.

Materials and Methods

Lignin Purification

Corn stover was pretreated at the National Renewable Energy Laboratory (NREL) using 70 kg NaOH/ton of corn stover with 1:12 solid:liquid ratio loading at 92° C. for two hours. The lignin residue was produced after disk refining (200 kwh/ODMT) using a 36 inch disk refiner (Sprout Waldon) at Andritz pilot plant (Springfield, Ohio) and enzymatic hydrolysis (48 mg CTec2 and 12 mg HTec2 per gram of cellulose for 36 hours). The enzymatic hydrolysis residue (namely alkali enzymatic lignin (AEL)) was then centrifuged to reduce the water content to approximately 20% solids. The received residual lignin was stored at −40° C. until use. Following a lignin precipitation method disclosed by He et al. (2017) in Lipid Production from Dilute Alkali corn stover lignin by Rhodococcus Strains, the AEL was further purified to remove residual carbohydrates. In short, the aqueous AEL slurry was brought to pH 12.5 (˜5:1 AEL to 2M NaOH), then the solution was centrifuged at 4000 rpm for 10 minutes to remove the solids containing undissolved carbohydrates. Then the lignin was precipitated from the filtrate by decreasing the pH to 3.0 with 2M H2SO4, centrifuged at 4000 rpm for 10 minutes to remove filtrate, and washed three times with 70° C. DI water. The resulting lignin was then freeze-dried using FREEZONE® 6 liter console freeze dry system (Labconco, Kansas City, Mo.) at −50° C. under 0.1-0.2 mBar vacuum for 72 hours. Tables 1 and 2 show the average purity in terms of weight percentage (wt %) of lignin samples derived from corn stover prior to and following precipitation purification treatment, respectively.

TABLE 1
Average purity of lignin samples derived from corn stover prior to
precipitation purification treatment in terms of weight percentage.

| | Lignin | Glucan | Xylan | Other |
|---|---|---|---|---|
| | (wt %) | (wt %) | (wt %) | (wt %) |
| Pre-Purification | 58.91 ± 5.67 | 22.15 ± 1.68 | 9.16 ± 0.71 | 9.78 ± 2.68 |
| Treatment | | | | |

TABLE 2
Average purity of lignin samples derived from corn stover following
precipitation purification treatment in terms of weight percentage.

| | Lignin | Glucan | Xylan |
|---|---|---|---|
| | (wt %) | (wt %) | (wt %) |
| Post-Purification | 95.11 ± 0.18 | 3.62 ± 0.16 | 1.27 ± 0.03 |
| Treatment | | | |

Structural carbohydrates and lignin composition of the resulting purified lignin samples were determined by compositional analysis. The sugar concentration of the lignin samples were determined by high performance liquid chromatography (HPLC) using an UltiMate 3000 System (Dionex Corporation, Sunnyvale, Calif.) equipped with a refractive index detector and using an AMINEX® HPX-87H column and guard assembly (Bio-Rad Laboratories, Inc., Hercules, Calif.).

Oxidative Depolymerization of Lignin

Oxidative procedures were carried out following the procedures previously disclosed by Ma et al. (2016) in Peracetic Acid Depolymerization of Biorefinery Lignin for Production of Selective Monomeric Phenolic Compounds. Specifically, the lignin was treated with a PAA dosage of 0.8 g PAA/g lignin with glacial acetic acid used to dilute the reaction mixture to 5% solid loading. Approximately 1 g of lignin was utilized in each reaction mixture. The reaction occurred at 60° C. for one hour while being mixed every ten minutes. Once the reaction was completed, the reaction mixture was centrifuged at 4000 rpm to remove unreacted solids and the supernatant was mixed with water at a 1:4 ratio to create an aqueous phase prior to bio-oil extraction. The bio-oil was extracted from the aqueous phase by mixing ethyl acetate at a 1:1 ratio three times. The ethyl acetate fractions were combined and dried under vacuum at 60° C. for 24 hours to obtain the bio-oil, which was then dissolved in ethanol and centrifuged to remove any undissolved solids.

Bio-Oil Characterization

Bio-oil yield was determined by weighing the total bio-oil content dissolved in ethanol and dividing by the starting lignin weight. The weight-average molecular weight (M_{w}) and the number-average molecular weight (Mₙ) of the raw corn stover lignin and PAA derived lignin bio-oils were determined using gel permeation chromatography (GPC). In this regard, an UltiMate3000 HPLC system (Dionex Corporation, Sunnyvale, Calif.) equipped with an Ultra Violet (UV) detector and Mixed-D PLgel column (5 m particle size, 300 mm×7.5 mm i.d., linear molecular weight range of 200 to 400,000 u) (Polymer Laboratories, Amherst, Mass.) was utilized. Separation was accomplished in a mobile phase of tetrahydrofuran (THF) at a flow rate of 0.5 ml minutes⁻¹, at 50° C. Elution profiles of materials were monitored at UV absorbance of 280 nm and calibrated using low molecular weight polystyrene standards (Product No. 48937, Sigma-Aldrich, St. Louis, Mo.). Polydispersity Index (PDI) was calculated using the equation: PDI=Mw/Mn.

The bio-oil was derivatized by first dissolving the bio-oil in 0.5 ml of pyridine then adding 0.5 ml of BSTFA and incubating at 50° C. for 30 min. Monomers were identified and quantified by GC/MS using a 7890B gas chromatography (GC) system coupled with a 5977B mass spectrometer (MS) with an HP-5 ms (60 m×0.32 mm) capillary column (Agilent Technologies, Santa Clara, Calif.). The temperature program started at 40° C. with a holding time of six minutes and increased to 240° C. at 4° C. min⁻¹ with a holding time of seven minutes, finally the temperature was raised to 280° C. at 20° C. min⁻¹ with a holding time of eight minutes. Helium was used as a carrier gas with a flow rate of 1.2 mL min⁻¹. Calibration curves were created using the commercially available pure compounds of: guaiacol, syringaldehyde, vanillin, and 4-propylphenol (Sigma Aldrich, St. Louis, Mo.).

Additionally, the total amount of phenolic compounds present in the bio-oil was estimated via microtiter-plated Folin-Ciocalteu assay. In this regard, reactions took place in 96-well microtiter plates and in each well 150 uL of water, 10 uL of Folin-Ciocalteu (F-C) reagent, and 2 μL of the proper dilution of test compound were added. The wells were mixed for five minutes and 30 uL of a 20% aqueous sodium carbonate solution was added to each well. The contents of the wells were then incubated at 45° C. for 30 minutes in a dry bath. The absorbance of the aliquots at 765 nm after the reaction with F-C reagent was measured against a blank using deionized water. The amount of total phenolics were quantified by correlating absorbances to standard curve generated from phenol standards at different concentrations.

Microbial Cultivation

USDA Agricultural Research Service Culture Collection (NRRL) provided Lactobacillus amylovorus (B-4540), Lactobacillus plantarum (NRRL B-4496) and Saccharomyces cerevisiae (NRRL Y-567) strains. Lactobacillus fermentum (0315-1) was provided by Dr. Joseph O. Rich from USDA (FIG. 1). The other lactic acid bacteria included Pediococcus pentosaceus, Enterococcus faecalis, Bacillus amyloliquefaciens, and Acetobacter pasteurianus, which were provided by Dr. Pat Heist from Ferm-Solutions™ (FIG. 1). Each microbe was grown on the recommended liquid media by NRRL with all lactic acid bacteria using M.R.S broth (Oxoid Microbiology Products, CM0359) and Saccharomyces cerevisiae using YPD media (Fisher BioReagents™, BP2469). All microbes had frozen cultures prepared by first growing each microbe in liquid culture at 180 rpm shaking speed for 12 hours at 37° C., except for Saccharomyces cerevisiae, which was grown at 32° C. The cultures' cells were pelletized via centrifugation and washed with sterile media. Then 500 μL of the washed cultures was added to 500 μL of sterilized 50% glycerol in a 2 mL cryovial and frozen at −80° C. until use.

USDA Agricultural Research Service Culture Collection (NRRL) provided the Escherichia coli (NRRL B-409), Lactobacillus amylovorus (B-4540), Saccharomyces cerevisiae (NRRL Y-567), Staphylococcus epidermidis (NRRL B-4268), and Bacillus subtilis (B-354) strains (FIG. 2). Each microbe was grown on the recommended liquid media by NRRL with E. coli using TGY media (tryptone 5 g/L, yeast extract 5 g/L, glucose 1 g/L, dipotassium phosphate 1 g/L), L. amylovorus using M.R.S broth (Oxoid, CM0359), Saccharomyces cerevisiae using YPD media (Fisher BioReagents™, BP2469), Subtilis epidermidis using nutrient broth (BD Difco™, 234000), and Bacillus subtilis using LB broth (Fisher BioReagents™, BP9723). All microbes had frozen cultures prepared by first growing each microbe in liquid culture at 180 rpm shaking speed for 12 hours at 37° C., except for Saccharomyces cerevisiae, which was grown at 32° C. The cultures' cells were pelletized via centrifugation and washed with sterile media. Then 500 μL of the washed cultures was added to 500 μL of sterilized 50% glycerol in a 2 mL cryovial and frozen at −80° C. until use.

Antimicrobial Assay

Frozen cultures of each microbe were first revived by adding the contents of the cryovials to liquid media and allowing growth for 12 hours at 180 rpm shaking speed at an incubation temperature of 37° C., except for Saccharomyces cerevisiae culture, which was incubated at 32° C. The cells were then pelletized, washed, and resuspended in fresh liquid media. To test for the bio-oil and sequential extraction fractions antimicrobial properties, each microbe was cultivated in 48-well plates and the OD₆₀₀ was monitored for 30 hours with time points taken at 0, 6, 10, 18, and 30 hours. These time points were previously found by the inventors to represent key points of microbial growth curves in unpublished data. All wells were brought to an OD₆₀₀ of 0.2 prior to growth, and the lignin bio-oils were tested at 0.5, 1.0, 1.5, 2.0, 2.5, 3, and 4 mg/ml concentrations. To facilitate the solubility of the bio-oils in media, all cultures had a final ethanol concentration of 5% (v/v). Two controls were used, one having the 5% ethanol concentration, and one having just microbes and media. The control of just microbes and media was used to ensure microbes were viable and actively growing. To determine how the bio-oils affected microbial growth, the percent change in OD₆₀₀ of the ethanol control during the exponential phase of growth was compared to the growth of the bio-oils at their different concentrations. This resulted in the percent decrease in growth (degree of inhibition) for each bio-oil at each concentration, with the formula described in Equation 1:

$$Degree of Inhibition ( \% ) = ( 1 - \frac{Max {OD}_{600} - Min {OD}_{600} of Growth with Oil}{Max {OD}_{600} - Min {OD}_{600} of Ethanol Control} ) * 100$$

Enzymatic Assay

To examine the effects of the lignin bio-oil on enzyme function during enzymatic saccharification, both α-amylase and glucoamylase were screened for activity while in the presence of the bio-oil at the highest concentration of 4 mg/ml. The dinitro salicylate (DNS) method was used to screen α-amylase activity. Prior to hydrolysis reaction, the α-amylase was suspended in phosphate buffer with and without the lignin-derived bio-oil at a concentration of 4 mg/ml, and allowed to interact for 30 minutes at ambient temperature. During the DNS assay, hydrolysis reactions took place in 2 ml Eppendorf tubes, where 0.5% (w/v) of potato starch in phosphate buffer (20 mM Sodium Phosphate with 6.7 mM Sodium Chloride, pH 6.9) was reacted with ˜1 unit of α-amylase for 10 minutes at 60° C. Additional bio-oil was added to the reaction mixture to ensure a constant concentration of 4 mg/ml. After the reaction DNS color reagent (5.3 M potassium sodium tartrate and 96 mM 3,5-Dinitrosalicylic acid solution) was added to the tubes and boiled for 15 minutes. The samples were immediately placed in an ice bath until they reached room temperature and then diluted with DI water prior to spectrophotometry. The absorbance at 540 nm was measured for the samples via spectrophotometry in 96 well plates. The difference in activity were determined by comparing the amount of sugar released in the samples with standard curves of maltose.

Glucoamylase inhibition was screened by measuring glucose content after hydrolysis using HPLC. Prior to hydrolysis reaction, the glucoamylase was suspended in acetate buffer (pH 5.6) with and without the lignin derived bio-oil at concentration of 4 mg/ml, and allowed to interact for 30 minutes at ambient temperature. For hydrolysis, the glucoamylase with and without the lignin-derived bio-oil was added to a 10 mg/ml maltose solution (in acetate buffer) and allowed to react for 30 minutes at 60° C. Additional bio-oil was added to the reaction mixture to ensure a constant concentration of 4 mg/ml. Afterwards, the reaction mixture was boiled for 15 minutes prior to glucose measurement. The glucose concentration released after hydrolysis was determined using an UltiMate 3000 HPLC system (Dionex Corporation, Sunnyvale, Calif., US) equipped with a refractive index detector and using an AMINEX® HPX-87H column and guard assembly (Bio-Rad, Hercules, Calif.). The difference in activity was then determined by comparing the amount of sugar released in the samples with the standard curves of glucose.

Results and Discussion

Bio-Oil Yield and Composition

The bio-oil produced from PAA depolymerization of alkali-extracted corn stover lignin was found to be 36.1±0.42 wt % of total starting lignin weight (Table 3), which represents the conversion yield of lignin to bio-oil.

TABLE 3
Average weight percentages of bio-oil, water soluble contents, and
unreacted solids of purified lignin samples represented in Table 2.

| Fraction | Average wt % |
|---|---|
| Ethyl Acetate Extracted Oil | 36.1 ± 0.42 |
| Remaining Water Soluble | 23.6 ± 0.89 |
| Unreacted Solids | 40.3 ± 1.43 |

Furthermore, as shown in Table 4 below, gas chromatograph-mass spectrometry (GCMS) analysis found 10 lignin derived monomers in the bio-oil that only accounted for 1.77 wt % of the bio-oil. Hydroxylated phenolics (i.e., hydroquinone) represented 47.2 wt % the total monomers detected.

TABLE 4
Lignin-derived monomers present within bio-oil extracted
from lignin samples, yield of each respective monomer with
respect to mg/ml of bio-oil, and weight percentage of the
bio-oil's total weight determined using GC/MS analysis.

| Compound | Yield (mg/ml) | Yield (wt %) |
|---|---|---|
| Hydroquinone | 0.69 | 0.83 |
| p-Coumaric acid | 0.30 | 0.36 |
| 2,6-Dimethoxyhydroquinone | 0.09 | 0.11 |
| Syringic acid | 0.09 | 0.11 |
| Phloroglucinol | 0.08 | 0.10 |
| 4-Hydroxybenzaldehyde | 0.07 | 0.09 |
| 4-Hydroxyacetophenone | 0.06 | 0.07 |
| Ferulic acid | 0.05 | 0.06 |
| 3-Ethylphenol | 0.01 | 0.02 |
| 2-Hydroxybenzyl alcohol | 0.01 | 0.01 |
| Total | 1.45 | 1.76 |

Although, the monomeric phenolic yields observed were very low, the Folin-Ciocalteu assay revealed that the bio-oil had a total phenolic content of 12.3 wt %. The overall phenolic content of the bio-oil is higher than the total monomer phenolic content found in the GC/MS results because the Folin-Ciocalteu assay is not limited to measuring monomeric phenolics. When GPC was performed, the average molecular weight of the compounds in the bio-oil was 2737 Daltons, and the range was from about 200-34884 Daltons, indicating there are much higher molecular weight compounds present in the bio-oil that are not lignin monomers. Since these higher molecular weight compounds are polymers, they cannot be easily identified by GC-MS. Nonetheless, due to the low monomer yields, it is believed that the antimicrobial activity provided by the lignin-derived bio-oil may be attributed primarily to the presence of dimers, trimers, and/or large oligomers within the bio-oil that have a size ranging from 200-34884 Daltons.

Inhibition of Bacterial Growth

As shown in the heat map of FIG. 1, the lignin-derived bio-oil was found to not inhibit the growth of Saccharomyces cerevisiae at any of the concentrations tested (0.5 mg/ml, 1 mg/ml, 1.5 mg/ml, 2 mg/ml, 2.5 mg/ml, 3 mg/ml, 4 mg/ml). Conversely, most of the LABs tested started showing growth reductions at around 2 mg/ml. Specifically, each of the LABs tested, except for Lactobacillus plantarum and Acetobacter pasteurianus, was found to inhibit growth by at least 60% at concentrations at 2 mg/ml (FIG. 1). With respect to Lactobacillus plantarum and Acetobacter pasteurianus, inhibition over 50% was not found until the bio-oil was present in concentrations of 3 mg/ml or more (FIG. 1). Growth of each LAB tested was found to be inhibited by 70% or greater when 4 mg/ml bio-oil was added (FIG. 1). Importantly, the bio-oil had more inhibition against Lactobacillus fermentum, which experienced a growth reduction of greater than 70% at concentrations ranging from 1-2.5 mg/ml and then over 90% at 3 mg/ml (FIG. 1). This is significant as the Lactobacillus fermentum tested has been found to be one of the most prolific strains causing stuck fermentations in the fuel corn ethanol industry. Therefore, the bio-oil was effective at reducing LAB growth while showing no effects on yeast growth. This provides evidence that the bio-oil may have ionophoric activity and could be used as an alternative to ionophoric antibiotics like virginiamycin in ethanol fermentation systems.

The lignin-derived bio-oil was also found to significantly inhibit Escherichia coli and Staphylococcus epidermidis at concentrations starting at 1.0 mg/ml and Bacillus subtilis at concentrations starting at 2.5 mg/ml, as shown in the heat map of FIG. 2.

Enzyme Activity in Presence of Bio-Oil

α-amylase was found to have a significant increase in activity, as measured by an increase in the amount of maltose released from hydrolysis of potato starch, in the presence of 4 mg/ml bio-oil as compared to the control, as shown in Table 5.

TABLE 5
Maltose content released from hydrolysis of potato starch
resulting from α-amylase enzymatic activity with
and without 4 gm/ml lignin-derived bio-oil treatment
based on 95% confidence interval using unpaired T-test.

| | Control Maltose | Bio-oil treatment | |
|---|---|---|---|
| | Content | Maltose Content | Unpaired T-Test |
| Enzyme | (mg/ml) | (mg/ml) | p-value |
| α-amylase | 0.990 ± 0.025 | 1.27 ± 0.022 | 0.0001 |

Conversely, glucoamylase had no significant difference in the amount of glucose released from hydrolysis of maltose in the presence of bio-oil as compared to the control, as shown in Table 6.

TABLE 6
Glucose content released from hydrolysis of maltose resulting
from glucoamylase enzymatic activity with and without
4 g/ml lignin-derived bio-oil treatment based on 95%
confidence interval using unpaired T-test.

| | Control Glucose | Bio-oil treatment | |
|---|---|---|---|
| | Content | Glucose Content | Unpaired T-Test |
| Enzyme | (mg/ml) | (mg/ml) | p-value |
| glucoamylase | 4.073 ± 0.074 | 4.323 ± 0.041 | 0.0947 |

The results of the enzymatic assays thus illustrate that enzymatic saccharification during corn ethanol fermentation will not be impacted by the bio-oil and may even be benefited by a slight increase in α-amylase activity.

All publications, patents, and patent applications mentioned in this specification are herein incorporated by reference to the same extent as if each individual publication, patent, or patent application was specifically and individually indicated to be incorporated by reference, including the references set forth in the following references list:

### REFERENCES

1. He, Y., et al., Lipid Production from Dilute Alkali Corn Stover Lignin by Rhodococcus Strains. ACS Sustainable Chemistry & Engineering, 2017. 5(3): p. 2302-2311.
2. Selig, M., N. Weiss, and Y. Ji, Enzymatic saccharification of lignocellulosic biomass. NREL Laboratory Analytical Procedure. 2008: Technical Report NREL/TP-510-42629, NREL, Colorado, USA. http://www.nrel.gov/docs/gen/fy08/42629.pdf.
3. Ma, R., et al., Peracetic Acid Depolymerization of Biorefinery Lignin for Production of Selective Monomeric Phenolic Compounds. Chemistry—A European Journal, 2016. 22(31): p. 10884-10891.
4. McClelland, D. J., et al., Functionality and molecular weight distribution of red oak lignin before and after pyrolysis and hydrogenation. Green Chemistry, 2017. 19(5): p. 1378-1389.
5. Magalhães, L. M., et al., Rapid microplate high-throughput methodology for assessment of Folin-Ciocalteu reducing capacity. Talanta, 2010. 83(2): p. 441-447.
6. Kazeem, M., J. Adamson, and I. Ogunwande, Modes of inhibition of α-amylase and α-glucosidase by aqueous extract of Morinda lucida Benth leaf BioMed research international, 2013. 2013.
7. Rich, J. O., et al., Resolving bacterial contamination of fuel ethanol fermentations with beneficial bacteria—An alternative to antibiotic treatment. Bioresource Technology, 2018. 247: p. 357-362.
8. M Bischoff, K., et al., Modeling Bacterial Contamination of Fuel Ethanol Fermentation. Vol. 103. 2009. 117-22.
9. Potters, G., Van Goethem, D. & Schutte, F., Promising Biofuel Resources: Lignocellulose and Algae, Nature Education, 2010. 3(9):14.
10. Cazacu, G., Capraru, M., Popa, V. I., 2013. Advances in Natural Polymers, Advanced Structured Materials. Springer, Berlin, Heidelberg, pp. 255-312.
11. Palmqvist E., Hahn-Hagerdal B. Fermentation of lignocellulosic hydrolysates. II: inhibitors and mechanisms of inhibition. Bioresour Technol. 2000a; 74:25-33.
12. Katahira, R.; Mittal, A.; McKinney, K.; Chen, X.; Tucker, M. P.; Johnson, D. K.; Beckham, G. T., Base-Catalyzed Depolymerization of Biorefinery Lignins. ACS Sustainable Chemistry & Engineering 2016, 4(3), 1474-1486.

What is claimed is:

1. A composition for inhibiting lactic acid bacteria growth in a media, comprising:
a decomposition product derived from an oxidative depolymerized lignin sample and including one or more phenolic constituents selected from the group consisting of (i) Hydroquinone, (ii) p-Coumaric acid, (iii) 2,6-Dimethoxyhydroquinone, (iv) Syringic acid, (v) Phloroglucinol, (vi) 4-Hydroxybenzaldehyde, (vii) 4-Hydroxyacetophenone, (viii) Ferulic acid, (ix) 3-Ethylphenol, and (x) 2-Hydroxybenzyl alcohol.

2. The composition of claim 1, wherein the one or more phenolic constituents is at least 1 wt % of an overall weight of the decomposition product.

3. The composition of claim 1, wherein a total phenolic content of the decomposition product is at least 10 wt % of an overall weight of the decomposition product.

4. The composition of claim 3, wherein the total phenolic content of the decomposition product is at least 12 wt % of the overall weight of the decomposition product.

5. The composition of claim 1, wherein the decomposition product is an oil.

6. The composition of claim 5, wherein the lignin sample is derived from corn stover.

7. The composition of claim 6, wherein the lignin sample is depolymerized using peracetic acid.

8. The composition of claim 1, wherein a phenolic content of the decomposition product is sufficient to inhibit the growth of one or more species of lactic acid bacteria within the media by at least 30% when the decomposition product is added to the media in a concentration of at least 2 mg/ml.

9. The composition of claim 8, wherein the one or more species of lactic acid bacteria include at least one of Lactobacillus plantarum, Lactobacillus amylovorus, Lactobacillus fermentum, Pediococcus pentosaceus, Enterococcus faecalis, Bacillus amyloliquefaciens, and Acetobacter pasteurianus.

10. The composition of claim 1, wherein a phenolic content of the decomposition product is sufficient to inhibit the growth Lactobacillus fermentum within the media by at least 70% when the decomposition product is added to the media in a concentration of at least 1 mg/ml.

11. The composition of claim 1, wherein a phenolic content of the decomposition product is such that the decomposition product does not inhibit growth of a species of yeast within the media when added to the media in a concentration of 4 mg/ml or less.

12. A composition for inhibiting bacteria growth in a media, comprising:
an oil derived from an oxidative depolymerized lignin sample;
wherein the oil includes one or more phenolic constituents and has a total phenolic content sufficient to inhibit the growth of Lactobacillus fermentum within the media by at least 70% when the decomposition product is added to the media in a concentration of at least 1 mg/ml.

13. A method for inhibiting bacteria growth, the method comprising the steps of:
implementing a decomposition product derived from an oxidative depolymerized lignin sample and including one or more phenolic constituents into a media contaminated by or at risk of being contaminated by lactic acid bacteria in a concentration sufficient to inhibit the growth of one or more species of lactic acid bacteria within the media.

14. The method of claim 13, wherein the one or more species of lactic acid bacteria includes at least one of Lactobacillus plantarum, Lactobacillus amylovorus, Lactobacillus fermentum, Pediococcus pentosaceus, Enterococcus faecalis, Bacillus amyloliquefaciens, and Acetobacter pasteurianus.

15. The method of claim 14, wherein the decomposition product is added to the media in a concentration sufficient to inhibit the growth of the one or more species of lactic acid bacteria by at least 30%.

16. The method of claim 13, wherein the media includes a species of yeast and the decomposition product is added to the media in a concentration which does not inhibit growth of the species of yeast.

17. The composition of claim 13, wherein the media includes Lactobacillus fermentum and the decomposition product is added in a concentration sufficient to inhibit the growth of Lactobacillus fermentum within the media by at least 70%.

18. The method of claim 13, wherein the one or more phenolic constituents include at least one of (i) Hydroquinone, (ii) p-Coumaric acid, (iii) 2,6-Dimethoxyhydroquinone, (iv) Syringic acid, (v) Phloroglucinol, (vi) 4-Hydroxybenzaldehyde, (vii) 4-Hydroxyacetophenone, (viii) Ferulic acid, (ix) 3-Ethylphenol, and (x) 2-Hydroxybenzyl alcohol.

19. The method of claim 13, wherein the decomposition product is an oil, and wherein the lignin sample is derived from corn stover and depolymerized using peracetic acid.

20. The method of claim 13, wherein the media is utilized in a fuel ethanol fermentation process.
